(19) United States
(12) Patent Application Publication
(10) Pub. No.: US 2026/0199024 A1
(43) Pub. Date: Jul. 16, 2026

(54) LANGUAGE-GUIDED NAVIGATION ASSISTANT FOR MEDICAL IMAGE ACQUISITION

(71) Applicants: Siemens Healthineers AG, Forchheim (DE); King's College London, London (GB)

(72) Inventors: Abdoul Aziz Amadou, London (GB); Vivek Singh, Princeton, NJ (US); Puneet Sharma, Princeton Junction, NJ (US); Young-Ho Kim, West Windsor, NJ (US); Kawal Rhode, Croydon (GB); Alistair Young, London (GB)

(21) Appl. No.: 19/326,819

(22) Filed: Sep. 12, 2025

Related U.S. Application Data
(60) Provisional application No. 63/745,492, filed on Jan. 15, 2025.

(51) Int. Cl. A61B 34/20; G06T 7/20; G06V 20/70; G16H 30/40

(52) U.S. Cl. CPC A61B 34/20; G06T 7/20; G06V 20/70; G16H 30/40; A61B 2034/2055; G06T 2207/30241

(57) ABSTRACT

Systems and methods for language-guided navigation assistance for medical image acquisition are provided. An initial image depicting a view of an image acquisition device at an initial position and a target image depicting a view of a target position are received. A trajectory between the initial position and the target position is determined based on the initial image and the target image. Text-based descriptions are generated for each step of the trajectory using a machine learning based vision-language model. One or more keyframes are selected from the trajectory based on the text-based descriptions. A particular natural language instruction for navigating the image acquisition device to a particular keyframe of the one or more keyframes to are generated. The natural language instructions are output.

### CROSS-REFERENCE TO RELATED APPLICATIONS

This application claims the benefit of U.S. Provisional Application No. 63/745,492, filed Jan. 15, 2025, the disclosure of which is herein incorporated by reference in its entirety.

### TECHNICAL FIELD

The present invention relates generally to medical image acquisition, and in particular to a language-guided navigation assistant for medical image acquisition.

### BACKGROUND

Acquisition of medical images is an intricate task that requires expert knowledge and extensive training. For instance, acquiring ultrasound images is dependent on the clinician's skill and experience, which can impact image quality and thus diagnostic outcomes. Similarly, acquiring cardiac MRI (magnetic resonance imaging) images involves a clinician manually defining planes to image certain anatomical structures. Providing clinicians with support during the image acquisition process can be beneficial in reducing examination time and errors due to user variability.

### BRIEF SUMMARY OF THE INVENTION

In accordance with one or more embodiments, systems and methods for language-guided navigation assistance for medical image acquisition are provided. An initial image depicting a view of an image acquisition device at an initial position and a target image depicting a view of a target position are received. A trajectory between the initial position and the target position is determined based on the initial image and the target image. Text-based descriptions are generated for each step of the trajectory using a machine learning based vision-language model. One or more keyframes are selected from the trajectory based on the text-based descriptions. A particular natural language instruction for navigating the image acquisition device to a particular keyframe of the one or more keyframes are generated. The natural language instructions are output.

In one embodiment, the image acquisition device is navigated to a current position according to a the particular natural language instruction to obtain a current image depicting a view of image acquisition device at the current position. A text-based description for the current image is generated using the machine learning based vision-language model. The text-based description for the current image is compared with the text-based description for the particular keyframe. It is determined whether the image acquisition device has reached the particular keyframe based on the comparing.

In one embodiment, in response to determining that the image acquisition device has not reached the particular keyframe, a next natural language instruction for navigating the image acquisition device to the particular keyframe is generated and the navigating, the generating the text-based description, the comparing, and the determining whether the image acquisition device has reached the particular keyframe are repeated using the next natural language instruction as the particular natural language instruction.

In one embodiment, in response to determining that the image acquisition device has reached the particular keyframe: a next natural language instruction for navigating the image acquisition device to the particular keyframe is generated and the navigating, the generating the text-based description, the comparing, and the determining whether the image acquisition device has reached the particular keyframe are repeated using the next natural language instruction as the particular natural language instruction and a next keyframe of the one or more keyframes as the particular keyframe.

In one embodiment, it is determined whether the image acquisition device has deviated from the trajectory based on the comparing. In response to determining that the image acquisition device has deviated from the trajectory, the determining the trajectory, the generating the text-based descriptions, the selecting the one or more keyframes, and the generating the particular natural language instruction are repeated using the current image as the initial image.

In one embodiment, a cosine similarity between the text-based description for the current image and the text-based description for the particular keyframe is determined.

In one embodiment, the trajectory comprises images between the initial position and the target position. The one or more keyframes are selected based on changes in contents of the images of the trajectory.

In one embodiment, the text-based descriptions comprise a list of structures, an indication of being a predefined view, and visible valves.

These and other advantages of the invention will be apparent to those of ordinary skill in the art by reference to the following detailed description and the accompanying drawings.

### BRIEF DESCRIPTION OF THE DRAWINGS

FIG. 1 shows a workflow for language-guided navigation of an image acquisition device for medical image acquisition, in accordance with one or more embodiments;

FIG. 2 shows a method for language-guided navigation of an image acquisition device for medical image acquisition, in accordance with one or more embodiments;

FIG. 3 shows an exemplary user interface for navigating the image acquisition device, in accordance with one or more embodiments;

FIG. 4 shows a table of metrics relating to navigation tasks completed in accordance with one or more embodiments described herein;

FIG. 5 shows a table of system usability scale scores per proficiency level for one or more embodiments described herein;

FIG. 6 shows an exemplary artificial neural network that may be used to implement one or more embodiments;

FIG. 7 shows a convolutional neural network that may be used to implement one or more embodiments;

FIG. 8 shows a data flow diagram for using a generative adversarial network according to one or more embodiments;

FIG. 9 shows a schematic structure of a recurrent machine learning model that may be used to implement one or more embodiments; and

FIG. 10 shows a high-level block diagram of a computer that may be used to implement one or more embodiments.

### DETAILED DESCRIPTION

The present invention generally relates to methods and systems for a language-guided navigation assistant for medical image acquisition. Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, it is to be understood that embodiments of the present invention may be performed within a computer system using data stored within the computer system. Further, reference herein to pixels of an image may refer equally to voxels of an image and vice versa.

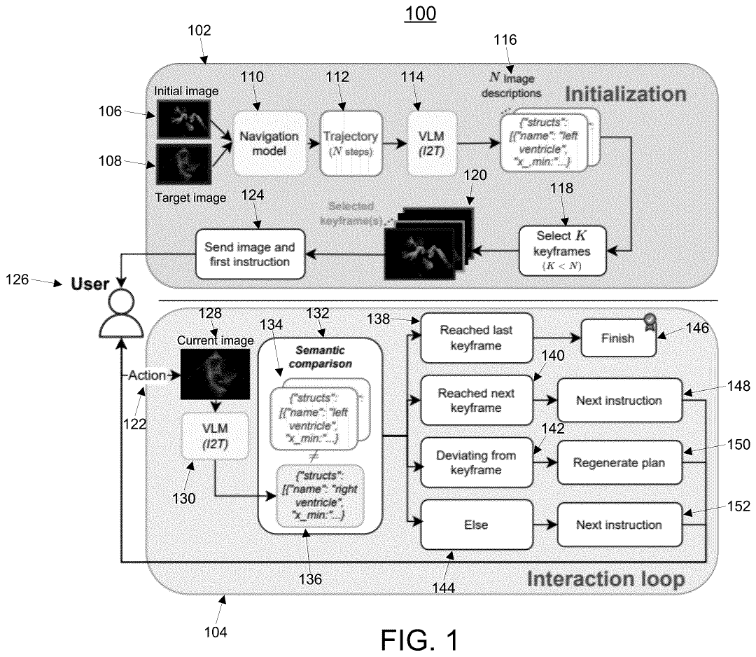

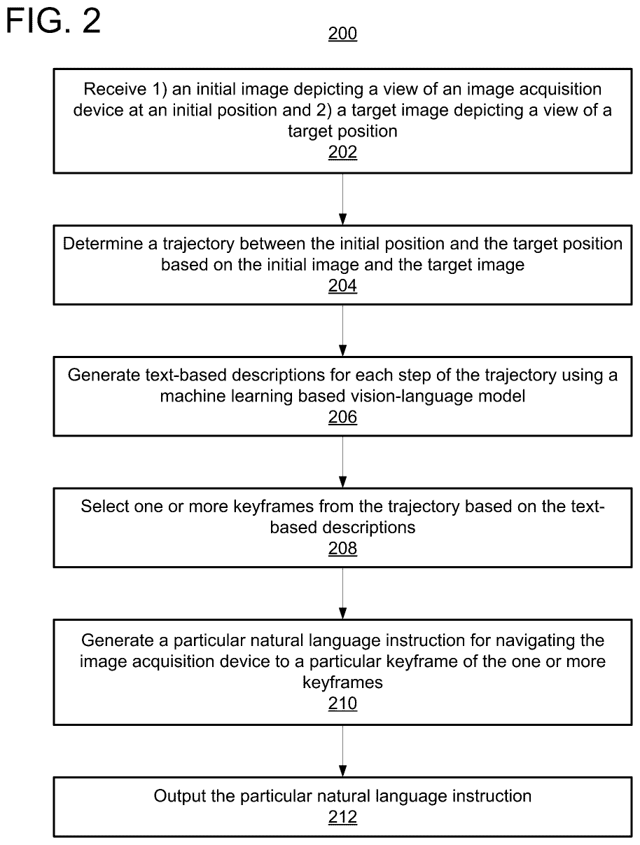

Embodiments described herein provide for a language-guided navigation system for medical image acquisition. FIG. 1 shows a workflow 100 for language-guided navigation of an image acquisition device for medical image acquisition, in accordance with one or more embodiments. Workflow 100 comprises two stages: initialization stage 102 and interaction loop 104. In general, during initialization stage 102, a trajectory 112 from an initial position to a target position of an image acquisition device is generated, keyframes 120 are selected 118 from trajectory 112, an initial natural language instruction is generated for navigating the image acquisition device from the initial position towards the target position, and the image and the initial instruction is sent 124 to user 126. During interaction loop 204, an action 122 is performed by user 126 to navigate the image acquisition device to a current position according to the initial natural-language instruction and, for each action/instruction, a semantic comparison 132 is performed between a text-based description 136 of a current image depicting a view of the image acquisition device at the current position and a text-based description 134 of a next keyframe is performed to assess progression of the navigation of the image acquisition device towards the target position. Based on semantic comparison 132, navigation is finished 146 when it is determined that the current position has reached the last of the selected keyframes 120, a next instruction 148 is generated when it is determined that the current position has reached a next keyframe 140 of the selected keyframes 120 and interaction loop 104 is repeated by performing action 122 according to the next instruction, the plan is regenerated 150 by repeating initialization stage 102 using the current position as the initial position when it is determined that the current position is deviating 142 from the keyframe, or a next instruction 142 for all other scenarios (i.e., else 144) and interaction loop 104 is repeated by performing action 122 according to the next instruction. Advantageously, language-guided navigation in accordance with embodiments described herein provide for reduced examination time and errors due to user variability. Further details of workflow 100 are described with respect to method 200 of FIG. 2.

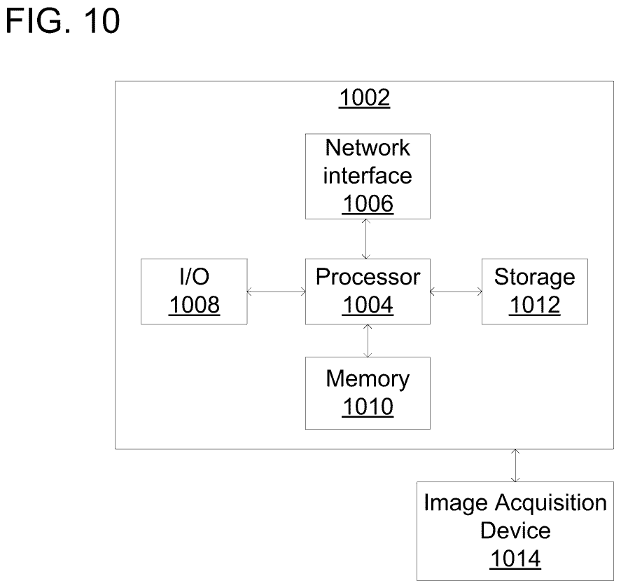

FIG. 2 shows a method 200 for language-guided navigation of an image acquisition device for medical image acquisition, in accordance with one or more embodiments. The steps and sub-steps of method 200 may be performed by one or more suitable computing devices, such as, e.g., computer 1002 of FIG. 10.

At step 202 of FIG. 2, 1) an initial image depicting a view of an image acquisition device at an initial position and 2) a target image depicting a view of the image acquisition device at a target position are received. In one example, as shown in workflow 100 of FIG. 1, the initial image is initial image 106 depicting a view of an image acquisition device at an initial position and the target image is target image 108 depicting a target position.

The initial image depicts a view of the medical image acquisition device at the initial position of a patient and represents the initial state of the medical image acquisition device. In one embodiment, the initial position is a predetermined position of a patient. The target image is a view of an anatomical object that the medical image acquisition device would depict at the target position and represents the target or goal state of the medical image acquisition device. The target image may be a pre-operative image of the anatomical object, obtained from any patient or a synthetic image generated from an image of another modality. The anatomical object may comprise, for example, organs, bones, vessels, tumors or other abnormalities, or any other anatomical object of interest. In one embodiment, the target image is selected from a library of images.

In one embodiment, the initial image and the target image are ultrasound images and the medical image acquisition device is a US (ultrasound) imaging device. In this embodiment, the initial position and the target position represent positions of a transducer or probe of the ultrasound imaging device. However, the initial image, the target image, and the medical image acquisition device may be of any other suitable modality, such as, e.g., MRI (magnetic resonance imaging), PET (positron emission tomography), SPECT (single photon emission computed tomography), CT (computed tomography), x-ray, or any other medical imaging modality or combinations of medical imaging modalities. The current image and/or the target image may be 2D (two dimensional) images and/or 3D (three dimensional) volumes, and may comprise a single input medical image or a plurality of input medical images.

The initial image and/or the target image may be received, for example, by directly receiving the images from the medical image acquisition device (e.g., image acquisition device 1014 of FIG. 10) as the images are acquired, by loading the images and/or text from a storage or memory of a computer system (e.g., storage 1012 or memory 1010 of computer 1002 of FIG. 10), or by receiving the images and/or text from a remote computer system (e.g., computer 1002 of FIG. 10). Such a computer system or remote computer system may comprise one or more patient databases, such as, e.g., an EHR (electronic health record), EMR (electronic medical record), PHR (personal health record), HIS (health information system), RIS (radiology information system), PACS (picture archiving and communication system), LIMS (laboratory information management system), or any other suitable database or system.

At step 204 of FIG. 2, a trajectory is determined between the initial position and the target position based on the initial image and the target image. In one example, as shown in workflow 100 of FIG. 1, trajectory 112 between the initial position and the target position is determined based on initial image 106 and target image 108 by navigation model 110.

The trajectory comprises N steps between the initial position and the target position, where N is any positive integer. In one embodiment, the steps of the trajectory are represented as images. However, the steps of the trajectory may be represented in any other suitable form. For example, the steps of the trajectory may be represented as vectors of latent features.

The trajectory may be determined using a machine learning based navigation model. In one embodiment, the machine learning based navigation model comprises a GCRL (goal-conditioned reinforcement learning) model. The GCRL model includes a dynamics model which predicts the steps of future states given a recurrent state and action. In one embodiment, the dynamics model is a RSSM (recurrent state-space model). The RSSM is adapted to add a latent adapter network, which takes the states (hₜ, zₜ) from the RSSM and predicts steps (e.g., images or latent feature vectors) from the VLM (vision-language model) utilized at step 206 of FIG. 2. This enables the text decoder of the VLM to decode the steps of the trajectory generated by the RSSM. The RSSM thus comprises the following machine learning based networks: 1) recurrent model hₜ=f_{φ}(hₜ₋₁, zₜ₋₁, aₜ₋₁), 2) representation model zₜ~q(zₜ|hₜ, oₜ) , 3) dynamics predictor [symbol] ~p_{φ}( [symbol] |hₜ, 4) image predictor [symbol] ~i_{φ}( [symbol] |hₜ, zₜ), and 5): p_{ψ}(oₜ)≈l_{φ}(hₜ, zₜ).

The GCRL model is implemented with an actor-critic architecture. Presented with a latent state representation concat (hₜ, zₜ) of the image generated by the RSSM and a target or goal image o_{g}, the actor network is trained to generate actions aₜ that lead to the target position. The critic network receives as input a triplet (oₜ, aₜ, o_{g}) and generates as output the probability density of reaching target image o_{g} when applying action aₜ given an image observation oₜ. During training, the critic network is trained using a contrastive loss between state-action and goal embeddings. Contrastive patient batching is used to stabilize critic learning, conjointly with a data augmented objective to enable better robustness and generalization. The actor network is trained to maximize the critic output.

At step 206 of FIG. 2, text-based descriptions are generated for each step of the trajectory using a machine learning based VLM. In one example, as shown in workflow 100 of FIG. 1, the text-based descriptions are the N image descriptions 116 generated by VLM 114 from trajectory 112.

The VLM receives as input the trajectory and generates as output the text-based description. The text-based descriptions may be represented in any suitable form. In one embodiment, the text-based descriptions are represented as JSON (JavaScript Object Notation) captions, which reduce output length and computation cost. Each JSON caption is a dictionary and may comprise, for example, three fields: 1) structs, which list detected anatomical structures or objects and their bounding boxes, 2) view, which indicate the standard view or “other”, and 3) valves, which enumerate visible cardiac valves.

The VLM comprises an image encoder and text decoders, denoted as p_{ψ}, q_{ψ} respectively. In one embodiment, the VLM is implemented as a query transformer (q-former). The VLM is pretrained with a frozen image encoder using three training objectives: a contrastive loss between image and text latents, and image captioning loss, and an image-text matching loss. The closeness in the latent space between image and text vectors due to the contrastive loss allows the VLM to decode vectors produced by the dynamics model (utilized at step 204 of FIG. 2). The image captioning loss teaches the VLM to produce descriptions of the images. The VLM may be implemented using any other suitable approach.

At step 208 of FIG. 2, one or more keyframes are selected from the trajectory based on the text-based descriptions. In one example, as shown in workflow 100 of FIG. 1, K keyframes 118 are selected based on image descriptions 116, where K is any positive integer less than N.

The keyframes represent waypoints between the initial position and the target position. In one embodiment, the keyframes are defined as steps where there was a significant change in the image contents of the trajectory with represent to the previous image of the trajectory. The keyframes may be selected using a rule-based approach. For example, given an image i at a timestep and an image j between the last keyframe k and i, image i is a keyframe if: 1) a structure present in image j disappears in i−1 and i, a structure absent in j appears in i−1 and i, or the view class in i−1 and i changes from the one in j (e.g., going from a two to a four-chamber view). The view class denotes a standard view or a class “other” representing all images that are not standard views.

At step 210 of FIG. 2, a particular natural language instruction is generated for navigating the image acquisition device to a particular keyframe of the one or more keyframes. Initially, the particular keyframe is a first keyframe of the one or more keyframe. In one example, as shown in workflow 100 of FIG. 1, a first instruction is generated and the initial image 106 and the first instruction are sent 124 to user 126.

The particular natural language instruction is an instruction for the user for guiding the image acquisition device to the particular keyframe. To generate the particular natural language instruction, the largest anatomical object (e.g., organs, bones, vessels, tumors, etc. or a portion thereof) is selected as the reference landmark. Then, rules are applied with respect to the largest anatomical object. For example, if the size of the largest anatomical object changes between two keyframes, the terms “increase” or “decrease” are used to describe the variation. In another example, if the position of the largest anatomical object between two keyframes changes, the horizontal and/or vertical changes are described. In a further example, if the view class changes to a standard (e.g., predefined) view, it is specified.

At step 212 of FIG. 2, the particular natural language instruction is output. For example, the particular natural language instruction can be output by displaying the particular natural language instruction on a display device of a computer system (e.g., I/O 1008 of computer 1002 of FIG. 10), storing the particular natural language instruction on a memory or storage of a computer system (e.g., memory 1010 or storage 1012 of computer 1002 of FIG. 10), or by transmitting the particular natural language instruction to a remote computer system (e.g., computer 1002 of FIG. 10).

In one embodiment, the image acquisition device is navigated according to the particular instruction. For example, as shown in FIG. 1, user 126 interacts with the image acquisition device to perform an action 122 according to the particular natural language instruction to navigate the image acquisition device to the particular keyframe. For example, the user may press a button on a computing device to automatically perform an action to navigate the image acquisition device. In another example, the user may manually navigate the image acquisition device according to the particular natural language instruction. The image acquisition device navigates to a current position and acquires a current image 128. Current image 128 depicts a view of the image acquisition device at the current position. A text-based description 136 of the current image 128 is generated by VLM 130. It should be understood that while individually represented for clarity, VLM 114 and 130 may be the same VLM. A comparison 132 is performed between the text-based description 136 of the current image 128 and the text-based description 134 of the particular keyframe. In one embodiment, comparison 132 is a semantic comparison, e.g., computed as the cosine similarity between text-based description 136 and text-based description 134. The semantic comparison may output a confidence score indicating the proximity of the image acquisition device at the current position to the target position. Comparison 132 may be performed according to any other suitable approach. It is then determined whether the image acquisition device has reached a condition (e.g., reached the last keyframe, reached the particular keyframe, deviating from the keyframe) based on the comparison 132.

In one embodiment, where it is determined that image acquisition device has reached 138 the particular keyframe based on the comparison 132 and the particular keyframe is the last keyframe, the workflow is finished 146 and the image acquisition device has reached the target position. For example, it is determined that the image acquisition device has reached the particular keyframe when the confidence score satisfies (e.g., is above) some threshold value. This occurs when the text-based descriptions 136 and 134 substantially match, for example, where all detected anatomical objects were present in both text-based descriptions 136 and 134, the sizes of all detected anatomical objects differed by less than, e.g., 25%, the positions of all detected anatomical objects are similar, and the standard views aligned.

In one embodiment, where it is determined that image acquisition device has reached 140 the particular keyframe based on the comparison 132 and the particular keyframe is not the last keyframe, a next natural language instruction 148 is generated to navigate the image acquisition device to a next keyframe of selected keyframes 120 and 148 and workflow 100 returns to user 126 performing action 122 according to the next instruction using the next keyframe as the particular keyframe.

In one embodiment, where it is determined that image acquisition device has deviated 142 from the trajectory towards the particular keyframe based on the comparison 132, initialization phase 102 is repeated using current image 128 as initial image 106 (i.e., steps 202-212 of FIG. 2 are repeated) to generate a new set of selected frames 120. For example, it may be determined that image acquisition device 142 is deviating from the trajectory when there are unexpected changes.

In one embodiment, in all other circumstances (i.e., else condition 144), for example, where the image acquisition device has not reached the particular keyframe (and is not deviating from the trajectory), a next natural language instruction 148 is generated and workflow 100 returns to user 126 performing action 122 according to the next instruction.

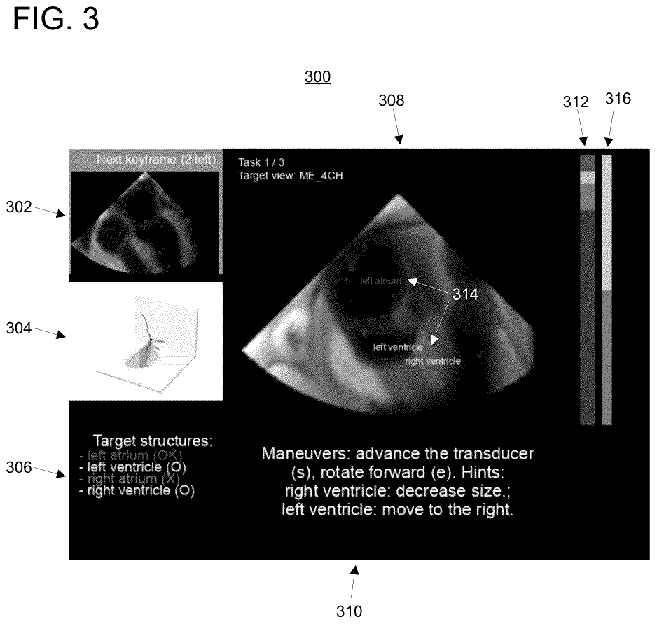

As the image acquisition device is navigated towards target image o_{g}, all applied actions are accumulated into an action vector. At each action, the action vector is updated, and the number of steps to reach the particular keyframe along the trajectory is computed. The two actions with the highest magnitude are selected as instructions and presented to the user. An exemplary user interface facilitating the user to navigate the image acquisition device is shown in FIG. 3.

FIG. 3 shows an exemplary user interface 300 for a user navigating the image acquisition device, in accordance with one or more embodiments. As shown in FIG. 3, user interface 300 shows a current image 308 depicting a view of the image acquisition device at a current position along a next keyframe 302, a 3D pose visualization 304, a list of target anatomical object 306 in the next keyframe, real-time anatomical labels 314 to aid recognition, natural language instructions 310, a confidence bar 312 showing proximity to the next keyframe, and a magnitude bar 316 showing the current magnitude of the applied motions. The anatomical labels 314 may be color coded to indicate status. For example, the anatomical labels 314 may be colored green indicating correctly positioned and size structures, yellow indicating structures that need adjustment, and gray indicating missing anatomical objects.

Embodiments described herein were experimentally validated using synthetic ultrasounds generated from CT datasets. The training data for the VLM was collected by using an agent moving the transducer randomly in a simulation environment. At every timestep, the image, its segmentation map and the corresponding action were stored. Around 3 million samples were collected. The dataset was resampled and rebalanced to 500 thousand samples, where 50% of the samples were not standard views, and the rest of the dataset was split equally between 5 standard view classes (ME (mid-esophageal) 2CH (two chamber), 4CH (four chamber), and 5CH (five chamber) views, AV (aortic valve) SAX (short axis) and LAX (long axis)).

The model's ability to describe images was evaluated. The IoU (intersection over union) was computed using predicted vs ground-truth bounding box coordinates for each anatomical landmark. The model reliably described the anatomical objects in the image, their location and accurately classified the views.

A user study was conducted to address the following questions: evaluate the usability of the ultrasound training application and assess how useful each visual and text cue was for learning. The following questions were addressed: Question 1) How useful are each one of the visual and text cues for learning to acquire views? and Question 2) How usable is the training application?

Study participants: All participants had education levels ranging from Master's to PhD and had basic knowledge of cardiac anatomy. They had various levels of exposure to cardiac ultrasound, ranging from no previous experience to being skilled at interpreting images. Ethics were approved by an institutional review board, and data were collected anonymously.

Study procedure: Participants logged into the application via a web interface. They were provided with a manual explaining the objective and the purpose of each element in the interface. They first completed a training mode to familiarize themselves with the application. Upon completion, they had to complete three navigation tasks to an ME 4CH view and three others to a ME 2CH view. Once finished, the participants were redirected to an online form to provide feedback on the application. More specifically, they provided feedback on the usefulness of each tool (on a scale of 1—poor to 5—good) and then answered questions from the SUS (system usability scale).

Data collection: Several metrics were recorded following the protocol completion by the participants. These include the success rate (if the participant reached the target view), the end position/angle errors with respect to the reference, the completion time for each task, the instruction adherence rate (i.e. the percentage of instructions followed during an episode) and questionnaire data.

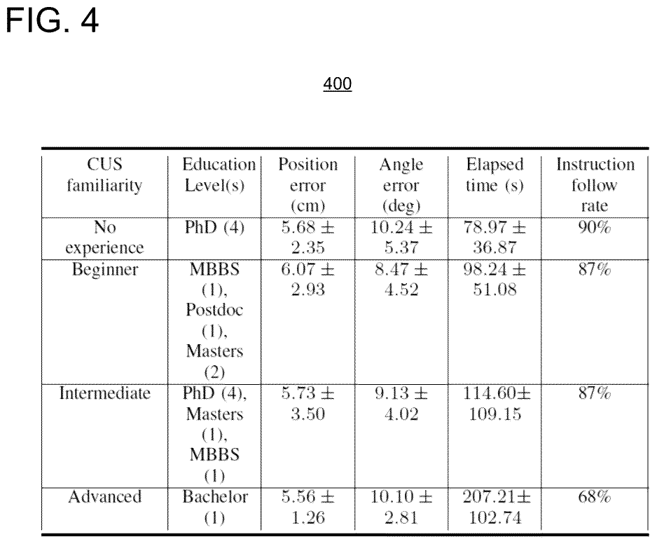

Study results: The metrics related to the navigation tasks completion per CUS (cardiac ultrasound) familiarity in accordance with one or more embodiments described herein are summarized in table 400 of FIG. 4. The instruction following rate decreased with increasing CUS familiarity. A Pearson correlation test between instruction adherence and completion time revealed a negative correlation between the two variables (Pearson r=−0.52, p<0.01), suggesting that following instructions helps complete tasks faster.

Question 1: Scores for each tool were obtained by summing the value of the rating from each participant. The higher the score, the higher the overall rating. The language instructions had the highest score, with a value of 67, followed by the 3D pose visualization (60), target structures labelling (59), the keyframes (56) and the Al confidence bar with the lowest score of 37. This highlights the necessity to provide real-time textual feedback and explanations to the users.

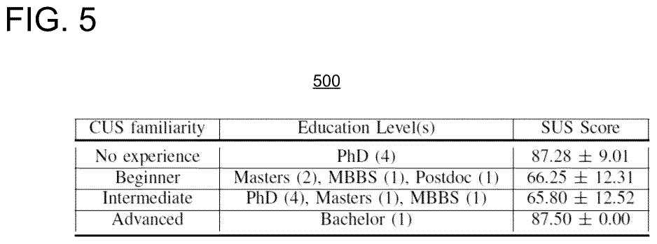

Question 2: The application's usability was also assessed using the SUS scale. A SUS score of 73.09±15.03 (meant std) was obtained. This corresponds to a B-score (good usability) following the Sauro and Lewis scale. Scores per proficiency level are detailed in table 500 of FIG. 5. Beginner and intermediate level users reported SUS scores significantly lower than users without experience (p<0.05), obtained with a one-sided permutation test). Users highlighted the need for technical support (SUS score of 35) and system complexity (score of 31) as improvement points, particularly among those in the beginner and intermediate groups. Additionally, the autoregressive caption generation from the VLM sometimes introduced a lag, as reported by some users. This could be alleviated using state-of-the-art decoding methods.

Embodiments described herein are described with respect to the claimed systems as well as with respect to the claimed methods. Features, advantages or alternative embodiments herein can be assigned to the other claimed objects and vice versa. In other words, claims and embodiments for the systems can be improved with features described or claimed in the context of the respective methods. In this case, the functional features of the method are implemented by physical units of the system.

Furthermore, certain embodiments described herein are described with respect to methods and systems utilizing trained machine learning models, as well as with respect to methods and systems for providing trained machine learning models. Features, advantages or alternative embodiments herein can be assigned to the other claimed objects and vice versa. In other words, claims and embodiments for providing trained machine learning models can be improved with features described or claimed in the context of utilizing trained machine learning models, and vice versa. In particular, datasets used in the methods and systems for utilizing trained machine learning models can have the same properties and features as the corresponding datasets used in the methods and systems for providing trained machine learning models, and the trained machine learning models provided by the respective methods and systems can be used in the methods and systems for utilizing the trained machine learning models.

In general, a trained machine learning model mimics cognitive functions that humans associate with other human minds. In particular, by training based on training data the machine learning model is able to adapt to new circumstances and to detect and extrapolate patterns. Another term for “trained machine learning model” is “trained function.”

In general, parameters of a machine learning model can be adapted by means of training. In particular, supervised training, semi-supervised training, unsupervised training, reinforcement learning and/or active learning can be used. Furthermore, representation learning (an alternative term is “feature learning”) can be used. In particular, the parameters of the machine learning models can be adapted iteratively by several steps of training. In particular, within the training a certain cost function can be minimized. In particular, within the training of a neural network the backpropagation algorithm can be used.

In particular, machine learning model described herein can comprise, for example, a neural network, a support vector machine, a decision tree and/or a Bayesian network, and/or the machine learning model can be based on, for example, k-means clustering, Q-learning, genetic algorithms and/or association rules. In particular, a neural network can be, e.g., a deep neural network, a convolutional neural network or a convolutional deep neural network. Furthermore, a neural network can be, e.g., an adversarial network, a deep adversarial network and/or a generative adversarial network.

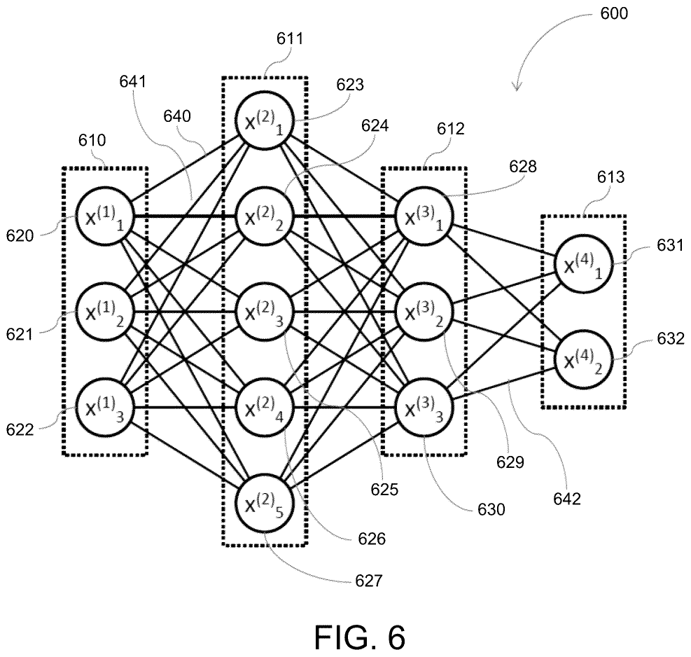

FIG. 6 shows an embodiment of an artificial neural network 600 that may be used to implement one or more machine learning models described herein. Alternative terms for “artificial neural network” are “neural network”, “artificial neural net” or “neural net”.

The artificial neural network 600 comprises nodes 620, . . . , 632 and edges 640, . . . , 642, wherein each edge 640, . . . , 642 is a directed connection from a first node 620, . . . , 632 to a second node 620, . . . , 632. In general, the first node 620, . . . , 632 and the second node 620, . . . , 632 are different nodes 620, . . . , 632, it is also possible that the first node 620, . . . , 632 and the second node 620, . . . , 632 are identical. For example, in FIG. 6 the edge 640 is a directed connection from the node 620 to the node 623, and the edge 642 is a directed connection from the node 630 to the node 632. An edge 640, . . . , 642 from a first node 620, . . . , 632 to a second node 620, . . . , 632 is also denoted as “ingoing edge” for the second node 620, . . . , 632 and as “outgoing edge” for the first node 620, . . . , 632.

In this embodiment, the nodes 620, . . . , 632 of the artificial neural network 600 can be arranged in layers 610, . . . , 613, wherein the layers can comprise an intrinsic order introduced by the edges 640, . . . , 642 between the nodes 620, . . . , 632. In particular, edges 640, . . . , 642 can exist only between neighboring layers of nodes. In the displayed embodiment, there is an input layer 610 comprising only nodes 620, . . . , 622 without an incoming edge, an output layer 613 comprising only nodes 631, 632 without outgoing edges, and hidden layers 611, 612 in-between the input layer 610 and the output layer 613. In general, the number of hidden layers 611, 612 can be chosen arbitrarily. The number of nodes 620, . . . , 622 within the input layer 610 usually relates to the number of input values of the neural network, and the number of nodes 631, 632 within the output layer 613 usually relates to the number of output values of the neural network.

In particular, a (real) number can be assigned as a value to every node 620, . . . , 632 of the neural network 600. Here, x⁽ⁿ⁾^{ᵢ} denotes the value of the i-th node 620, . . . , 632 of the n-th layer 610, . . . , 613. The values of the nodes 620, . . . , 622 of the input layer 610 are equivalent to the input values of the neural network 600, the values of the nodes 631, 632 of the output layer 613 are equivalent to the output value of the neural network 600. Furthermore, each edge 640, . . . , 642 can comprise a weight being a real number, in particular, the weight is a real number within the interval [−1, 1] or within the interval [0, 1]. Here, w^{(m,n)}^{_{i,j}} denotes the weight of the edge between the i-th node 620, . . . , 632 of the m-th layer 610, . . . , 613 and the j-th node 620, . . . , 632 of the n-th layer 610, . . . , 613. Furthermore, the abbreviation w⁽ⁿ⁾^{_{i,j}} is defined for the weight w^{(n,n+1)}^{_{i,j}}.

In particular, to calculate the output values of the neural network 600, the input values are propagated through the neural network. In particular, the values of the nodes 620, . . . , 632 of the (n+1)-th layer 610, . . . , 613 can be calculated based on the values of the nodes 620, . . . , 632 of the n-th layer 610, . . . , 613 by

$${x}^{{( n + 1 )}_{j}} = f ( {\sum }_{i} {x}^{{( n )}_{i}} \cdot {w}^{{( n )}_{i , j}} ) .$$

Herein, the function f is a transfer function (another term is “activation function”). Known transfer functions are step functions, sigmoid function (e.g., the logistic function, the generalized logistic function, the hyperbolic tangent, the Arctangent function, the error function, the smoothstep function) or rectifier functions. The transfer function is mainly used for normalization purposes.

In particular, the values are propagated layer-wise through the neural network, wherein values of the input layer 610 are given by the input of the neural network 600, wherein values of the first hid-den layer 611 can be calculated based on the values of the input layer 610 of the neural network, wherein values of the second hidden layer 612 can be calculated based in the values of the first hidden layer 611, etc.

In order to set the values w^{(m,n)}^{_{i,j}} for the edges, the neural network 600 has to be trained using training data. In particular, training data comprises training input data and training output data (denoted as tᵢ). For a training step, the neural network 600 is applied to the training input data to generate calculated output data. In particular, the training data and the calculated output data comprise a number of values, said number being equal with the number of nodes of the output layer.

In particular, a comparison between the calculated output data and the training data is used to recursively adapt the weights within the neural network 600 (backpropagation algorithm). In particular, the weights are changed according to

$${w}^{{′ ( n )}_{i , j}} = {w}^{{( n )}_{i , j}} - γ \cdot {δ}^{{( n )}_{j}} \cdot {x}^{{( n )}_{i}}$$

wherein γ is a learning rate, and the numbers δ⁽ⁿ⁾^{ⱼ} can be recursively calculated as

$${δ}^{{( n )}_{j}} = ( {\sum }_{k} {δ}^{{( n + 1 )}_{k}} \cdot {w}^{{( n + 1 )}_{j , k}} ) \cdot {f}^{′} ( {\sum }_{i} {x}^{{( n )}_{i}} \cdot {w}^{{( n )}_{i , j}} )$$

based on δ⁽ⁿ⁺¹⁾^{ⱼ}, if the (n+1)-th layer is not the output layer, and

$${δ}^{{( n )}_{j}} = ( {x}^{{( n + 1 )}_{j}} - {t}^{{( n + 1 )}_{j}} ) \cdot {f}^{′} ( {x}^{{( n )}_{i}} \cdot {w}^{{( n )}_{i j}} )$$

if the (n+1)-th layer is the output layer 613, wherein f′ is the first derivative of the activation function, and t⁽ⁿ⁺¹⁾^{ⱼ} is the comparison training value for the j-th node of the output layer 613.

A convolutional neural network is a neural network that uses a convolution operation instead of general matrix multiplication in at least one of its layers (so-called “convolutional layer”). In particular, a convolutional layer performs a dot product of one or more convolution kernels with the convolutional layer's input data/image, wherein the entries of the one or more convolution kernels are the parameters or weights that are adapted by training. In particular, one can use the Frobenius inner product and the ReLU activation function. A convolutional neural network can comprise additional layers, e.g., pooling layers, fully connected layers, and normalization layers.

By using convolutional neural networks input images can be processed in a very efficient way, because a convolution operation based on different kernels can extract various image features, so that by adapting the weights of the convolution kernel the relevant image features can be found during training. Furthermore, based on the weight-sharing in the convolutional kernels less parameters need to be trained, which prevents overfitting in the training phase and allows to have faster training or more layers in the network, improving the performance of the network.

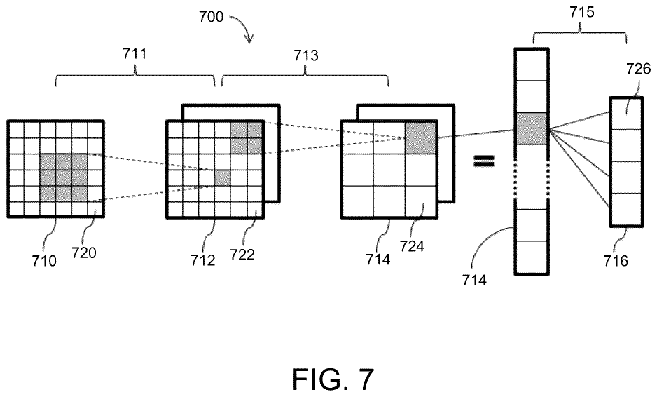

FIG. 7 shows an embodiment of a convolutional neural network 700 that may be used to implement one or more machine learning models described herein. In the displayed embodiment, the convolutional neural network 700 comprises an input node layer 710, a convolutional layer 711, a pooling layer 713, a fully connected layer 714 and an output node layer 716, as well as hidden node layers 712, 714. Alternatively, the convolutional neural network 700 can comprise several convolutional layers 711, several pooling layers 713 and several fully connected layers 715, as well as other types of layers. The order of the layers can be chosen arbitrarily, usually fully connected layers 715 are used as the last layers before the output layer 716.

In particular, within a convolutional neural network 700 nodes 720, 722, 724 of a node layer 710, 712, 714 can be considered to be arranged as a d-dimensional matrix or as a d-dimensional image. In particular, in the two-dimensional case the value of the node 720, 722, 724 indexed with i and j in the n-th node layer 710, 712, 714 can be denoted as x(n)[i, j]. However, the arrangement of the nodes 720, 722, 724 of one node layer 710, 712, 714 does not have an effect on the calculations executed within the convolutional neural network 700 as such, since these are given solely by the structure and the weights of the edges.

A convolutional layer 711 is a connection layer between an anterior node layer 710 (with node values x(n−1)) and a posterior node layer 712 (with node values x(n)). In particular, a convolutional layer 711 is characterized by the structure and the weights of the incoming edges forming a convolution operation based on a certain number of kernels. In particular, the structure and the weights of the edges of the convolutional layer 711 are chosen such that the values x(n) of the nodes 722 of the posterior node layer 712 are calculated as a convolution x(n)=K*x(n−1) based on the values x(n−1) of the nodes 720 anterior node layer 710, where the convolution * is defined in the two-dimensional case as

$${x}_{k}^{( n )} [ i , j ] = ( K ⋆ {x}^{( n - 1 )} ) [ i , j ] = {\sum }_{{i}^{′}} {\sum }_{{j}^{′}} K [ {i}^{′} , {j}^{′} ] \cdot {x}^{( n - 1 )} [ i - {i}^{′} , i - {j}^{′} ] .$$

Here the kernel K is a d-dimensional matrix (in this embodiment, a two-dimensional matrix), which is usually small compared to the number of nodes 720, 722 (e.g., a 3×3 matrix, or a 5×5 matrix). In particular, this implies that the weights of the edges in the convolution layer 711 are not independent, but chosen such that they produce said convolution equation. In particular, for a kernel being a 3×3 matrix, there are only 9 independent weights (each entry of the kernel matrix corresponding to one independent weight), irrespectively of the number of nodes 720, 722 in the anterior node layer 710 and the posterior node layer 712.

In general, convolutional neural networks 700 use node layers 710, 712, 714 with a plurality of channels, in particular, due to the use of a plurality of kernels in convolutional layers 711. In those cases, the node layers can be considered as (d+1)-dimensional matrices (the first dimension indexing the channels). The action of a convolutional layer 711 is then a two-dimensional example defined as

$${x}^{{( n )}_{b}} [ i , j ] = {\sum }_{a} {K}_{a , b} * {x}^{{( n - 1 )}_{a}} [ i , j ] = {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ]$$

where x⁽ⁿ⁻¹⁾^{ₐ} corresponds to the a-th channel of the anterior node layer 710, x⁽ⁿ⁾^{_{b}} corresponds to the b-th channel of the posterior node layer 712 and K_{a,b} corresponds to one of the kernels. If a convolutional layer 711 acts on an anterior node layer 710 with A channels and outputs a posterior node layer 712 with B channels, there are A·B independent d-dimensional kernels K_{a,b}.

In general, in convolutional neural networks 700 activation functions are used. In this embodiment ReLU (acronym for “Rectified Linear Units”) is used, with R(z)=max(0, z), so that the action of the convolutional layer 711 in the two-dimensional example is

$${x}^{{( n )}_{b}} [ i , j ] = R ( {\sum }_{a} ( {K}_{a , b} * {x}^{{( n - 1 )}_{a}} ) [ i , j ] ) = R ( {\sum }_{a} {\sum }_{{i}^{′}} {\sum }_{{j}^{′}} {K}_{a , b} [ {i}^{′} , {j}^{′} ] \cdot {x}^{{( n - 1 )}_{a}} [ i - {i}^{′} , j - {j}^{′} ] )$$

It is also possible to use other activation functions, e.g., ELU (acronym for “Exponential Linear Unit”), LeakyReLU, Sigmoid, Tanh or Softmax.

In the displayed embodiment, the input layer 710 comprises 36 nodes 720, arranged as a two-dimensional 6×6 matrix. The first hidden node layer 712 comprises 72 nodes 722, arranged as two two-dimensional 6×6 matrices, each of the two matrices being the result of a convolution of the values of the input layer with a 3×3 kernel within the convolutional layer 711. Equivalently, the nodes 722 of the first hidden node layer 712 can be interpreted as arranged as a three-dimensional 2×6×6 matrix, wherein the first dimension correspond to the channel dimension.

The advantage of using convolutional layers 711 is that spatially local correlation of the input data can exploited by enforcing a local connectivity pattern between nodes of adjacent layers, in particular by each node being connected to only a small region of the nodes of the preceding layer.

A pooling layer 713 is a connection layer between an anterior node layer 712 (with node values x(n−1)) and a posterior node layer 714 (with node values x(n)). In particular, a pooling layer 713 can be characterized by the structure and the weights of the edges and the activation function forming a pooling operation based on a non-linear pooling function f. For example, in the two-dimensional case the values x(n) of the nodes 724 of the posterior node layer 714 can be calculated based on the values x(n−1) of the nodes 722 of the anterior node layer 712 as

$${x}^{{( n )}_{b}} [ i , j ] = f ( {x}^{( n - 1 )} [ {id}_{1} , {jd}_{2} ] , … , {x}^{{( n - 1 )}_{b}} [ ( i + 1 ) {d}_{1} - 1 , ( j + 1 ) {d}_{2} - 1 ] )$$

In other words, by using a pooling layer 713 the number of nodes 722, 724 can be reduced, by re-placing a number d1·d2 of neighboring nodes 722 in the anterior node layer 712 with a single node 722 in the posterior node layer 714 being calculated as a function of the values of said number of neighboring nodes. In particular, the pooling function f can be the max-function, the average or the L2-Norm. In particular, for a pooling layer 713 the weights of the incoming edges are fixed and are not modified by training.

The advantage of using a pooling layer 713 is that the number of nodes 722, 724 and the number of parameters is reduced. This leads to the amount of computation in the network being reduced and to a control of overfitting.

In the displayed embodiment, the pooling layer 713 is a max-pooling layer, replacing four neighboring nodes with only one node, the value being the maximum of the values of the four neighboring nodes. The max-pooling is applied to each d-dimensional matrix of the previous layer; in this embodiment, the max-pooling is applied to each of the two two-dimensional matrices, reducing the number of nodes from 72 to 18.

In general, the last layers of a convolutional neural network 700 are fully connected layers 715. A fully connected layer 715 is a connection layer between an anterior node layer 714 and a posterior node layer 716. A fully connected layer 713 can be characterized by the fact that a majority, in particular, all edges between nodes 714 of the anterior node layer 714 and the nodes 716 of the posterior node layer are present, and wherein the weight of each of these edges can be adjusted individually.

In this embodiment, the nodes 724 of the anterior node layer 714 of the fully connected layer 715 are displayed both as two-dimensional matrices, and additionally as non-related nodes (indicated as a line of nodes, wherein the number of nodes was reduced for a better presentability). This operation is also denoted as “flattening”. In this embodiment, the number of nodes 726 in the posterior node layer 716 of the fully connected layer 715 smaller than the number of nodes 724 in the anterior node layer 714. Alternatively, the number of nodes 726 can be equal or larger.

Furthermore, in this embodiment the Softmax activation function is used within the fully connected layer 715. By applying the Softmax function, the sum the values of all nodes 726 of the output layer 716 is 1, and all values of all nodes 726 of the output layer 716 are real numbers between 0 and 1. In particular, if using the convolutional neural network 700 for categorizing input data, the values of the output layer 716 can be interpreted as the probability of the input data falling into one of the different categories.

In particular, convolutional neural networks 700 can be trained based on the backpropagation algorithm. For preventing overfitting, methods of regularization can be used, e.g., dropout of nodes 720, . . . , 724, stochastic pooling, use of artificial data, weight decay based on the L1 or the L2 norm, or max norm constraints.

According to an aspect, the machine learning model may comprise one or more residual networks (ResNet). In particular, a ResNet is an artificial neural network comprising at least one jump or skip connection used to jump over at least one layer of the artificial neural network. In particular, a ResNet may be a convolutional neural network comprising one or more skip connections respectively skipping one or more convolutional layers. According to some examples, the ResNets may be represented as m-layer ResNets, where m is the number of layers in the corresponding architecture and, according to some examples, may take values of 34, 50, 101, or 152. According to some examples, such an m-layer ResNet may respectively comprise (m−2)/2 skip connections.

A skip connection may be seen as a bypass which directly feeds the output of one preceding layer over one or more bypassed layers to a layer succeeding the one or more bypassed layers. Instead of having to directly fit a desired mapping, the bypassed layers would then have to fit a residual mapping “balancing” the directly fed output.

Fitting the residual mapping is computationally easier to optimize than the directed mapping. What is more, this alleviates the problem of vanishing/exploding gradients during optimization upon training the machine learning models: if a bypassed layer runs into such problems, its contribution may be skipped by regularization of the directly fed output. Using ResNets thus brings about the advantage that much deeper networks may be trained.

A generative adversarial model (an acronym is GA model) comprises a generative function and a discriminative function, wherein the generative function creates synthetic data, and the discriminative function distinguishes between synthetic and real data. By training the generative function and/or the discriminative function on the one hand the generative function is configured to create synthetic data which is incorrectly classified by the discriminative function as real, on the other hand the discriminative function is configured to distinguish between real data and synthetic data generated by the generative function. In the notion of game theory, a generative adversarial model can be interpreted as a zero-sum game. The training of the generative function and/or of the discriminative function is based, in particular, on the minimization of a cost function.

By using a GA model, based on a set of training data synthetic data can be generated that has the same characteristics as the training data set. The training of the GA model can be based on data not being annotated (unsupervised learning), so that there is low effort in training a GA model.

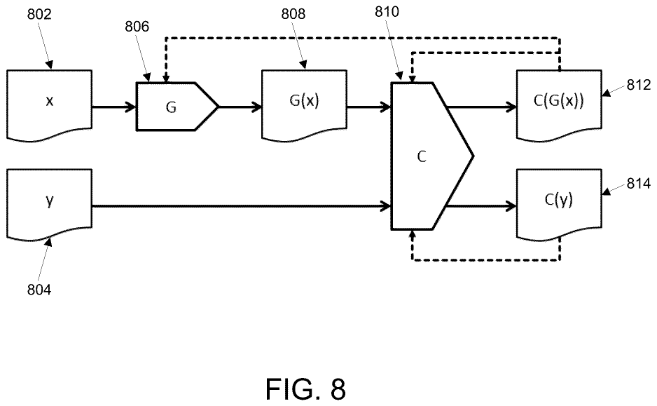

FIG. 8 shows a data flow diagram according to an embodiment for using a generative adversarial network for creating synthetic output data G(x) 808 based on input data x 802 that is indistinguishable from real output data y 804, in accordance with one or more embodiments. The synthetic output data G(x) 808 has the same structure as the real output data y 804, but its content is not derived from real world data.

The generative adversarial network comprises a generator function G 806 and a classifier function C 810 which are trained jointly. The task of the generator function G 806 is to provide realistic synthetic output data G(x) 808 based on input data x 802, and the task of the classifier function C 810 is to distinguish between real output data y 804 and synthetic output data G(x) 808. In particular, the output of the classifier function C 810 is a real number between 0 and 1 corresponding to the probability of the input value being real data, so that an ideal classifier function would calculate an output value of C(y) 814≈1 for real data y 804 and C(G(x)) 812≈0 for synthetic data G(x) 808.

Within the training process, parameters of the generator function G 806 are adapted so that the synthetic output data G(x) 808 has the same characteristics as real output data y 804, so that the classifier function C 810 cannot distinguish between real and synthetic data anymore. At the same time, parameters of the classifier function C 810 are adapted so that it distinguishes between real and synthetic data in the best possible way. Here, the training relies on pairs comprising input data x 802 and the corresponding real output data y 804. Within a single training step, the generator function G 806 is applied to the input data x 802 for generating synthetic output data G(x) 808. Furthermore, the classifier function C 810 is applied to the real output data y 804 for generating a first classification result C(y) 814. Additionally, the classifier function C 810 is applied to the synthetic output data G(x) 808 for generating a second classification result C(G(x)) 812.

Adapting the parameters of the generative function G 806 and the classifier function C 810 is based on minimizing a cost function by using the backpropagation algorithm, respectively. In this embodiment, the cost function K_{C} for the classifier function C 810 is K_{C}∝−BCE(C(y), 1)−BCE(C(G(x), 0), wherein BCE denotes the binary cross entropy defined as BCE(z, z′)=z′·log(z)+(1−z′)·log(1−z). By using this cost function, both wrongly classifying real output data as synthetic (indicated by C(y)=0) and wrongly classifying synthetic output data as real (indicated as C(G(x)) 812≈1) increases the cost function K_{C} to be minimized. Furthermore, the cost function K_{G} for the generator function G 806 is K_{G}∝−BCE(C(G(x), 1)=−log(C(G(x). By using this cost function, correctly classified synthetic output data (indicated as C(G(x)) 812≈0) leads to an increase of the cost function K_{G} to be minimized.

In particular, a recurrent machine learning model is a machine learning model whose output does not only depend on the input value and the parameters of the machine learning model adapted by the training process, but also on a hidden state vector, wherein the hidden state vector is based on previous inputs used on for the recurrent machine learning model. In particular, the recurrent machine learning model can comprise additional storage states or additional structures that incorporate time delays or comprise feedback loops.

In particular, the underlying structure of a recurrent machine learning model can be a neural network, which can be denoted as recurrent neural network. Such a recurrent neural network can be described as an artificial neural network where connections between nodes form a directed graph along a temporal sequence. In particular, a recurrent neural network can be interpreted as directed acyclic graph. In particular, the recurrent neural network can be a finite impulse recurrent neural network or an infinite impulse recurrent neural network (wherein a finite impulse network can be unrolled and replaced with a strictly feedforward neural network, and an infinite impulse network cannot be unrolled and replaced with a strictly feedforward neural network).

In particular, training a recurrent neural network can be based on the BPTT algorithm (acronym for “backpropagation through time”), on the RTRL algorithm (acronym for “real-time recurrent learning”) and/or on genetic algorithms.

By using a recurrent machine learning model input data comprising sequences of variable length can be used. In particular, this implies that the method cannot be used only for a fixed number of input datasets (and needs to be trained differently for every other number of input datasets used as input), but can be used for an arbitrary number of input datasets. This implies that the whole set of training data, independent of the number of input datasets contained in different sequences, can be used within the training, and that training data is not reduced to training data corresponding to a certain number of successive input datasets.

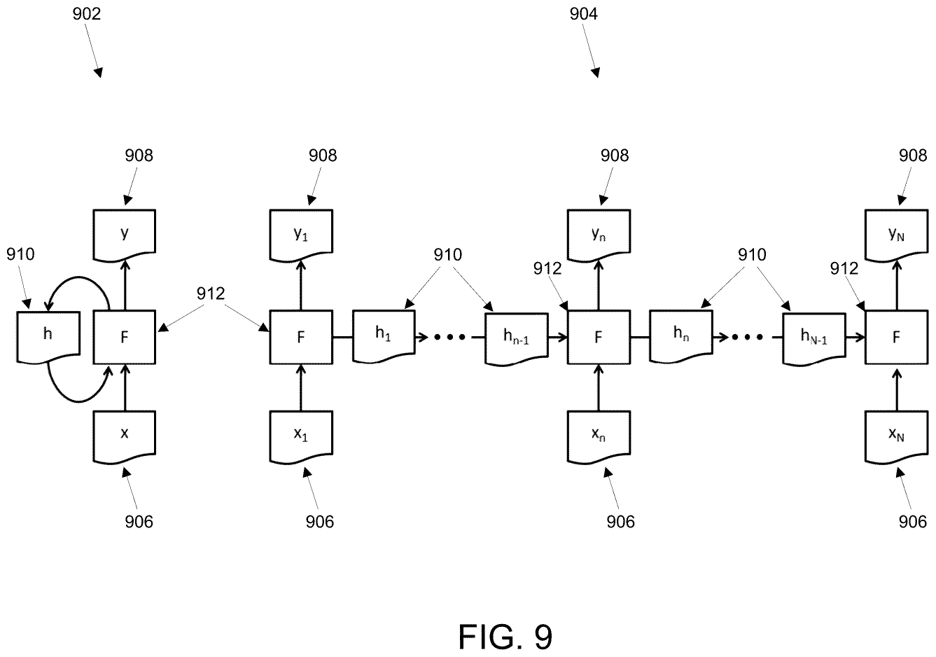

FIG. 9 shows the schematic structure of a recurrent machine learning model F, both in a recurrent representation 902 and in an unfolded representation 904, that may be used to implement one or more machine learning models described herein. The recurrent machine learning model takes as input several input datasets x, x₁, . . . , x_{N} 906 and creates a corresponding set of output datasets y, y₁, . . . , y_{N} 908. Furthermore, the output depends on a so-called hidden vector h, h₁, . . . , h_{N} 910, which implicitly comprises information about input datasets previously used as input for the recurrent machine learning model F 912. By using these hidden vectors h, h₁, . . . , h_{N} 910, a sequentiality of the input datasets can be leveraged.

In a single step of the processing, the recurrent machine learning model F 912 takes as input the hidden vector hₙ₋₁ created within the previous step and an input dataset xₙ. Within this step, the recurrent machine learning model F generates as output an updated hidden vector hₙ and an output dataset yₙ. In other words, one step of processing calculates (yₙ, hₙ)=F(xₙ, hₙ₋₁), or by splitting the recurrent machine learning model F 912 into a part F(y) calculating the output data and F(h) calculating the hidden vector, one step of processing calculates yₙ=F^{(y)}(xₙ, hₙ₋₁) and hₙ=F^{(h)}(xₙ, hₙ₋₁). For the first processing step, h₀ can be chosen randomly or filled with all entries being zero. The parameters of the recurrent machine learning model F 912 that were trained based on training datasets before do not change between the different processing steps.

In particular, the output data and the hidden vector of a processing step depend on all the previous input datasets used in the previous steps. yₙ=F^{(y)}(xₙ, F^{(h)}(xₙ₋₁, hₙ₋₂)) and hₙ=F^{(h)}(xₙ, F^{(h)}(xₙ₋₁, hₙ₋₂).

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatuses, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatuses, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 1 or 2. Certain steps or functions of the methods and workflows described herein, including one or more of the steps or functions of FIG. 1 or 2, may be performed by a server or by another processor in a network-based cloud-computing system. Certain steps or functions of the methods and workflows described herein, including one or more of the steps of FIG. 1 or 2, may be performed by a client computer in a network-based cloud computing system. The steps or functions of the methods and workflows described herein, including one or more of the steps of FIG. 1 or 2, may be performed by a server and/or by a client computer in a network-based cloud computing system, in any combination.

Systems, apparatuses, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 1 or 2, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

A high-level block diagram of an example computer 1002 that may be used to implement systems, apparatuses, and methods described herein is depicted in FIG. 10. Computer 1002 includes a processor 1004 operatively coupled to a data storage device 1012 and a memory 1010. Processor 1004 controls the overall operation of computer 1002 by executing computer program instructions that define such operations. The computer program instructions may be stored in data storage device 1012, or other computer readable medium, and loaded into memory 1010 when execution of the computer program instructions is desired. Thus, the method and workflow steps or functions of FIG. 1 or 2 can be defined by the computer program instructions stored in memory 1010 and/or data storage device 1012 and controlled by processor 1004 executing the computer program instructions. For example, the computer program instructions can be implemented as computer executable code programmed by one skilled in the art to perform the method and workflow steps or functions of FIG. 1 or 2. Accordingly, by executing the computer program instructions, the processor 1004 executes the method and workflow steps or functions of FIG. 1 or 2. Computer 1002 may also include one or more network interfaces 1006 for communicating with other devices via a network. Computer 1002 may also include one or more input/output devices 1008 that enable user interaction with computer 1002 (e.g., display, keyboard, mouse, speakers, buttons, etc.).

Processor 1004 may include both general and special purpose microprocessors, and may be the sole processor or one of multiple processors of computer 1002. Processor 1004 may include one or more central processing units (CPUs), for example. Processor 1004, data storage device 1012, and/or memory 1010 may include, be supplemented by, or incorporated in, one or more application-specific integrated circuits (ASICs) and/or one or more field programmable gate arrays (FPGAs).

Data storage device 1012 and memory 1010 each include a tangible non-transitory computer readable storage medium. Data storage device 1012, and memory 1010, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1008 may include peripherals, such as a printer, scanner, display screen, etc. For example, input/output devices 1008 may include a display device such as a cathode ray tube (CRT) or liquid crystal display (LCD) monitor for displaying information to the user, a keyboard, and a pointing device such as a mouse or a trackball by which the user can provide input to computer 1002.

An image acquisition device 1014 can be connected to the computer 1002 to input image data (e.g., medical images) to the computer 1002. It is possible to implement the image acquisition device 1014 and the computer 1002 as one device. It is also possible that the image acquisition device 1014 and the computer 1002 communicate wirelessly through a network. In a possible embodiment, the computer 1002 can be located remotely with respect to the image acquisition device 1014.

Any or all of the systems, apparatuses, and methods discussed herein may be implemented using one or more computers such as computer 1002.

One skilled in the art will recognize that an implementation of an actual computer or computer system may have other structures and may contain other components as well, and that FIG. 10 is a high level representation of some of the components of such a computer for illustrative purposes.

Independent of the grammatical term usage, individuals with male, female or other gender identities are included within the term.

The foregoing Detailed Description is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

The following is a list of non-limiting illustrative embodiments disclosed herein:

Illustrative embodiment 1. A computer-implemented method comprising: receiving 1) an initial image depicting a view of an image acquisition device at an initial position and 2) a target image depicting a view of a target position; determining a trajectory between the initial position and the target position based on the initial image and the target image; generating text-based descriptions for each step of the trajectory using a machine learning based vision-language model; selecting one or more keyframes from the trajectory based on the text-based descriptions; generating a particular natural language instruction for navigating the image acquisition device to a particular keyframe of the one or more keyframes; and outputting the particular natural language instruction.

Illustrative embodiment 2. The computer-implemented method of illustrative embodiment 1, further comprising: navigating the image acquisition device to a current position according to the particular natural language instruction to obtain a current image depicting a view of image acquisition device at the current position; generating a text-based description for the current image using the machine learning based vision-language model; comparing the text-based description for the current image with the text-based description for the particular keyframe; and determining whether the image acquisition device has reached the particular keyframe based on the comparing.

Illustrative embodiment 3. The computer-implemented method of illustrative embodiment 2, further comprising: in response to determining that the image acquisition device has not reached the particular keyframe: generating a next natural language instruction for navigating the image acquisition device to the particular keyframe; and repeating the navigating, the generating the text-based description, the comparing, and the determining whether the image acquisition device has reached the particular keyframe using the next natural language instruction as the particular natural language instruction.

Illustrative embodiment 4. The computer-implemented method of any one of illustrative embodiments 2-3, further comprising: in response to determining that the image acquisition device has reached the particular keyframe: generating a next natural language instruction for navigating the image acquisition device to the particular keyframe; and repeating the navigating, the generating the text-based description, the comparing, and the determining whether the image acquisition device has reached the particular keyframe using the next natural language instruction as the particular natural language instruction and a next keyframe of the one or more keyframes as the particular keyframe.

Illustrative embodiment 5. The computer-implemented method of any one of illustrative embodiments 2-4, further comprising: determining whether the image acquisition device has deviated from the trajectory based on the comparing; and in response to determining that the image acquisition device has deviated from the trajectory, repeating the determining the trajectory, the generating the text-based descriptions, the selecting the one or more keyframes, and the generating the particular natural language instruction using the current image as the initial image.

Illustrative embodiment 6. The computer-implemented method of any one of illustrative embodiments 2-5, wherein comparing the text-based description for the current image with the text-based description for the particular keyframe comprises: determining a cosine similarity between the text-based description for the current image and the text-based description for the particular keyframe.

Illustrative embodiment 7. The computer-implemented method of any one of illustrative embodiments 1-6, wherein the trajectory comprises images between the initial position and the target position.

Illustrative embodiment 8. The computer-implemented method of illustrative embodiment 7, wherein selecting one or more keyframes from the trajectory based on the text-based descriptions comprises: selecting the one or more keyframes based on changes in contents of the images of the trajectory.

Illustrative embodiment 9. The computer-implemented method of any one of illustrative embodiments 1-8, wherein the text-based descriptions comprise a list of structures, an indication of being a predefined view, and visible valves.

Illustrative embodiment 10. An apparatus comprising: means for receiving 1) an initial image depicting a view of an image acquisition device at an initial position and 2) a target image depicting a view of a target position; means for determining a trajectory between the initial position and the target position based on the initial image and the target image; means for generating text-based descriptions for each step of the trajectory using a machine learning based vision-language model; means for selecting one or more keyframes from the trajectory based on the text-based descriptions; means for generating a particular natural language instruction for navigating the image acquisition device to a particular keyframe of the one or more keyframes; and means for outputting the particular natural language instruction.

Illustrative embodiment 11. The apparatus of illustrative embodiment 10, further comprising: means for navigating the image acquisition device to a current position according to the particular natural language instruction to obtain a current image depicting a view of image acquisition device at the current position; means for generating a text-based description for the current image using the machine learning based vision-language model; means for comparing the text-based description for current image with the text-based description for the particular keyframe; and means for determining whether the image acquisition device has reached the particular keyframe based on the comparing.

Illustrative embodiment 12. The apparatus of illustrative embodiment 11, further comprising: in response to determining that the image acquisition device has not reached the particular keyframe: means for generating a next natural language instruction for navigating the image acquisition device to the particular keyframe; and means for repeating the navigating, the generating the text-based description, the comparing, and the determining whether the image acquisition device has reached the particular keyframe using the next natural language instruction as the particular natural language instruction.

Illustrative embodiment 13. The apparatus of any one of illustrative embodiments 11-12, further comprising: in response to determining that the image acquisition device has reached the particular keyframe: means for generating a next natural language instruction for navigating the image acquisition device to the particular keyframe; and means for repeating the navigating, the generating the text-based description, the comparing, and the determining whether the image acquisition device has reached the particular keyframe using the next natural language instruction as the particular natural language instruction and a next keyframe of the one or more keyframes as the particular keyframe.

Illustrative embodiment 14. The apparatus of any one of illustrative embodiments 11-13, further comprising: means for determining whether the image acquisition device has deviated from the trajectory based on the comparing; and in response to determining that the image acquisition device has deviated from the trajectory, means for repeating the determining the trajectory, the generating the text-based descriptions, the selecting the one or more keyframes, and the generating the particular natural language instruction using the current image as the initial image.

Illustrative embodiment 15. The apparatus of any one of illustrative embodiments 11-14, wherein the means for comparing the text-based description for the current image with the text-based description for the particular keyframe comprises: means for determining a cosine similarity between the text-based description for the current image and the text-based description for the particular keyframe.

Illustrative embodiment 16. A non-transitory computer-readable storage medium comprising instructions which, when executed by a computer, cause the computer to carry out operations comprising: receiving 1) an initial image depicting a view of an image acquisition device at an initial position and 2) a target image depicting a view of a target position; determining a trajectory between the initial position and the target position based on the initial image and the target image; generating text-based descriptions for each step of the trajectory using a machine learning based vision-language model; selecting one or more keyframes from the trajectory based on the text-based descriptions; generating a particular natural language instruction for navigating the image acquisition device to a particular keyframe of the one or more keyframes; and outputting the particular natural language instruction.

Illustrative embodiment 17. The non-transitory computer-readable storage medium of illustrative embodiment 16, the operations further comprising: navigating the image acquisition device to a current position according to the particular natural language instruction to obtain a current image depicting a view of image acquisition device at the current position; generating a text-based description for the current image using the machine learning based vision-language model; comparing the text-based description for current image with the text-based description for the particular keyframe; and determining whether the image acquisition device has reached the particular keyframe based on the comparing.

Illustrative embodiment 18. The non-transitory computer-readable storage medium of any one of illustrative embodiments 16-17, wherein the trajectory comprises images between the initial position and the target position.

Illustrative embodiment 19. The non-transitory computer-readable storage medium of illustrative embodiment 18, wherein selecting one or more keyframes from the trajectory based on the text-based descriptions comprises: selecting the one or more keyframes based on changes in contents of the images of the trajectory.

Illustrative embodiment 20. The non-transitory computer-readable storage medium of any one of illustrative embodiments 16-19, wherein the text-based descriptions comprise a list of structures, an indication of being a predefined view, and visible valves.

1. A computer-implemented method comprising:
receiving 1) an initial image depicting a view of an image acquisition device at an initial position and 2) a target image depicting a view of a target position;
determining a trajectory between the initial position and the target position based on the initial image and the target image;
generating text-based descriptions for each step of the trajectory using a machine learning based vision-language model;
selecting one or more keyframes from the trajectory based on the text-based descriptions;
generating a particular natural language instruction for navigating the image acquisition device to a particular keyframe of the one or more keyframes; and
outputting the particular natural language instruction.

2. The computer-implemented method of claim 1, further comprising:
navigating the image acquisition device to a current position according to the particular natural language instruction to obtain a current image depicting a view of image acquisition device at the current position;
generating a text-based description for the current image using the machine learning based vision-language model;
comparing the text-based description for the current image with the text-based description for the particular keyframe; and
determining whether the image acquisition device has reached the particular keyframe based on the comparing.

3. The computer-implemented method of claim 2, further comprising:
in response to determining that the image acquisition device has not reached the particular keyframe:
generating a next natural language instruction for navigating the image acquisition device to the particular keyframe; and
repeating the navigating, the generating the text-based description, the comparing, and the determining whether the image acquisition device has reached the particular keyframe using the next natural language instruction as the particular natural language instruction.

4. The computer-implemented method of claim 2, further comprising:
in response to determining that the image acquisition device has reached the particular keyframe:
generating a next natural language instruction for navigating the image acquisition device to the particular keyframe; and
repeating the navigating, the generating the text-based description, the comparing, and the determining whether the image acquisition device has reached the particular keyframe using the next natural language instruction as the particular natural language instruction and a next keyframe of the one or more keyframes as the particular keyframe.

5. The computer-implemented method of claim 2, further comprising:
determining whether the image acquisition device has deviated from the trajectory based on the comparing; and
in response to determining that the image acquisition device has deviated from the trajectory, repeating the determining the trajectory, the generating the text-based descriptions, the selecting the one or more keyframes, and the generating the particular natural language instruction using the current image as the initial image.

6. The computer-implemented method of claim 2, wherein comparing the text-based description for the current image with the text-based description for the particular keyframe comprises:
determining a cosine similarity between the text-based description for the current image and the text-based description for the particular keyframe.

7. The computer-implemented method of claim 1, wherein the trajectory comprises images between the initial position and the target position.

8. The computer-implemented method of claim 7, wherein selecting one or more keyframes from the trajectory based on the text-based descriptions comprises:
selecting the one or more keyframes based on changes in contents of the images of the trajectory.

9. The computer-implemented method of claim 1, wherein the text-based descriptions comprise a list of structures, an indication of being a predefined view, and visible valves.

10. An apparatus comprising:
means for receiving 1) an initial image depicting a view of an image acquisition device at an initial position and 2) a target image depicting a view of a target position;
means for determining a trajectory between the initial position and the target position based on the initial image and the target image;
means for generating text-based descriptions for each step of the trajectory using a machine learning based vision-language model;
means for selecting one or more keyframes from the trajectory based on the text-based descriptions;
means for generating a particular natural language instruction for navigating the image acquisition device to a particular keyframe of the one or more keyframes; and
means for outputting the particular natural language instruction.

11. The apparatus of claim 10, further comprising:
means for navigating the image acquisition device to a current position according to the particular natural language instruction to obtain a current image depicting a view of image acquisition device at the current position;
means for generating a text-based description for the current image using the machine learning based vision-language model;
means for comparing the text-based description for current image with the text-based description for the particular keyframe; and
means for determining whether the image acquisition device has reached the particular keyframe based on the comparing.

12. The apparatus of claim 11, further comprising:
in response to determining that the image acquisition device has not reached the particular keyframe:
means for generating a next natural language instruction for navigating the image acquisition device to the particular keyframe; and
means for repeating the navigating, the generating the text-based description, the comparing, and the determining whether the image acquisition device has reached the particular keyframe using the next natural language instruction as the particular natural language instruction.

13. The apparatus of claim 11, further comprising:
in response to determining that the image acquisition device has reached the particular keyframe:
means for generating a next natural language instruction for navigating the image acquisition device to the particular keyframe; and
means for repeating the navigating, the generating the text-based description, the comparing, and the determining whether the image acquisition device has reached the particular keyframe using the next natural language instruction as the particular natural language instruction and a next keyframe of the one or more keyframes as the particular keyframe.

14. The apparatus of claim 11, further comprising:
means for determining whether the image acquisition device has deviated from the trajectory based on the comparing; and
in response to determining that the image acquisition device has deviated from the trajectory, means for repeating the determining the trajectory, the generating the text-based descriptions, the selecting the one or more keyframes, and the generating the particular natural language instruction using the current image as the initial image.

15. The apparatus of claim 11, wherein the means for comparing the text-based description for the current image with the text-based description for the particular keyframe comprises:
means for determining a cosine similarity between the text-based description for the current image and the text-based description for the particular keyframe.

16. A non-transitory computer-readable storage medium comprising instructions which, when executed by a computer, cause the computer to carry out operations comprising:
receiving 1) an initial image depicting a view of an image acquisition device at an initial position and 2) a target image depicting a view of a target position;
determining a trajectory between the initial position and the target position based on the initial image and the target image;
generating text-based descriptions for each step of the trajectory using a machine learning based vision-language model;
selecting one or more keyframes from the trajectory based on the text-based descriptions;
generating a particular natural language instruction for navigating the image acquisition device to a particular keyframe of the one or more keyframes; and
outputting the particular natural language instruction.

17. The non-transitory computer-readable storage medium of claim 16, the operations further comprising:
navigating the image acquisition device to a current position according to the particular natural language instruction to obtain a current image depicting a view of image acquisition device at the current position;
generating a text-based description for the current image using the machine learning based vision-language model;
comparing the text-based description for the current image with the text-based description for the particular keyframe; and
determining whether the image acquisition device has reached the particular keyframe based on the comparing.

18. The non-transitory computer-readable storage medium of claim 16, wherein the trajectory comprises images between the initial position and the target position.

19. The non-transitory computer-readable storage medium of claim 18, wherein selecting one or more keyframes from the trajectory based on the text-based descriptions comprises:
selecting the one or more keyframes based on changes in contents of the images of the trajectory.

20. The non-transitory computer-readable storage medium of claim 16, wherein the text-based descriptions comprise a list of structures, an indication of being a predefined view, and visible valves.
